(19) United States
(12) Patent Application Publication
(10) Pub. No.: US 2018/0360627 A1
(43) Pub. Date: Dec. 20, 2018

(54) STENT WITH HELICAL GROOVE

(71) Applicant: Cook Medical Technologies LLC, Bloomington, IN (US)

(72) Inventor: Shuo Yang, West Lafayette, IN (US)

(21) Appl. No.: 16/010,594

(22) Filed: Jun. 18, 2018

Related U.S. Application Data
(60) Provisional application No. 62/521,699, filed on Jun. 19, 2017.

(51) Int. Cl. A61F 2/88; A61F 2/89

(52) U.S. Cl. CPC A61F 2/88; A61F 2/89; A61F 2210/0014; A61F 2/07; A61F 2230/006; A61F 2230/0056; A61F 2240/001

(57) ABSTRACT

Disclosed herein is an intraluminal support device having a groove formed therein, and a method of making such a support device. The device has an improved architecture in which the ring-shaped segments are joined by connectors that are spaced at even intervals from the groove in order to maintain a consistent radial force profile and stabilize the device.

### CROSS REFERENCE TO RELATED APPLICATIONS

This application claims the benefit of U.S. Provisional Patent Application No. 62/521,699, filed on Jun. 19, 2017, the entire contents of which is hereby incorporated by reference.

### BACKGROUND

The present application generally relates to medical devices. More particularly, the present application relates to implantable intraluminal support devices having internal geometries effective to modulate the pattern of blood flow therethrough.

Stents and other implantable medical devices are deployed to the vasculature of patients in need thereof in order to support vessels and permit substantially normal flow of blood therethrough. However, the mismatch at the junction of an intraluminal device with that of the bare vessel can cause anomalies in blood flow pattern, leading to stagnant regions at the junction, where solid particulate can build up, further disrupting flow patterns.

Although devices that have geometric features that redirect fluid flow through a tubular lumen in a body vessel have been previously described, in general these have been achieved by using custom mandrels to impose the shape of the flow diverting element, such as a groove, a ridge, or other geometric feature, on an existing medical implant or device. Although such a workflow may yield acceptable results in certain cases, it will be appreciated by those of skill in the art that the physics of medical device construction impacts how the device will behave during loading, delivery, and while providing the intended therapeutic effect. Therefore, a device purpose-built for the task to be accomplished, rather than a modification to an existing device, may be advantageous.

It has been a challenge to develop an implantable device that modulates blood flow therethrough with an architecture that imparts structural advantages to the device both when compressed and expanded.

### SUMMARY

In one aspect, the present disclosure provides an intraluminal support device for implantation into a lumen of a body vessel. The intraluminal support device may include a tubular body having a first end extending to a second end to define a length. The tubular body may define a longitudinal axis and a lumen. The tubular body may have an inner surface and an outer surface. The first end and the second end may be open and in fluid communication with the lumen. The tubular body may include a plurality of rings disposed axially along the longitudinal axis, and each ring may be connected to an adjacent ring by at least one connector segment. The tubular body may be radially expandable from a compressed state to an expanded state, and a plurality of the connector segments may be arranged in a helical pattern having a helical angle. The intraluminal support device may include a helical groove formed in the tubular body which defines a helical ridge extending into the lumen. The helical groove may have a helical angle equal to the helical angle of the helical pattern, and the connector segments of the helical pattern may be spaced substantially constantly from the groove along the length of the tubular body.

In another aspect, the present disclosure provides an intraluminal support device for implantation into a lumen of a body vessel. The intraluminal support device may include a tubular body having a first end extending to a second end and defining a length. The tubular body defines a longitudinal axis and a lumen. The tubular body has an inner surface and an outer surface. The first end and the second end may be open and in fluid communication with the lumen. The tubular body may include a plurality of hoop rings and flex rings disposed axially and alternately along the longitudinal axis. Each flex ring may be connected to an adjacent hoop ring by at least one connector segment. The tubular body may be radially expandable from a compressed state to an expanded state. A plurality of the connector segments may be arranged in a helical pattern having a helical angle. The intraluminal support device including a helical groove may be formed in the tubular body and may define a helical ridge extending into the lumen. The helical groove may have a helical angle equal to the helical angle of the helical pattern. The connector segments of the helical pattern may be spaced substantially constantly from the groove along the length of the tubular body.

In another aspect, the present disclosure provides method of making an intraluminal support device for implantation into a body vessel. The method includes a step of placing the intraluminal support device over a cylindrical mandrel having a helical groove formed thereon. The intraluminal support device may include a tubular body having a first end extending to a second end and defining a length. The tubular body defines a longitudinal axis and a lumen and has an inner surface and an outer surface. The first end and the second end are open and in fluid communication with the lumen. The tubular body may be radially expandable from a compressed state to an expanded state. The tubular body may include a plurality of rings disposed axially along the longitudinal axis, each of the rings being connected to one another by at least one connector segment to form a plurality of rings. A plurality of the connector segments may be arranged in a helical pattern. The connector segments of the helical pattern may be spaced substantially constantly from the groove along the length of the tubular body. The method may also include a step of securing the intraluminal support device to the cylindrical mandrel such that the helical groove of the mandrel is bounded by the connector segments of the helical pattern. The method may include fitting a portion of each ring into the helical groove. The method may include heat-setting the intraluminal support device such that the outer surface includes a helical groove formed therein and extending into the lumen.

Further objects, features and advantages of this system will become readily apparent to persons skilled in the art after a review of the following description, with reference to the drawings and claims that are appended to and form a part of this specification.

### BRIEF DESCRIPTION OF THE DRAWINGS

FIG. 1 is a side view of a cylindrical grooved mandrel for making a medical device in accordance with the principles of the present invention;

FIG. 2 is a flattened schematic view of a medical device constructed in accordance with an embodiment of the present invention;

FIG. 3 is a side view of a portion of a medical device constructed in accordance with the principles of the present disclosure;

FIG. 4 is a perspective view of a portion of a medical device constructed in accordance with the principles of the present disclosure;

FIG. 5 is a partial cross-sectional view of a medical device constructed in accordance with the principles of the present disclosure; and

FIG. 6 is a flattened schematic view of a medical device constructed in accordance with another embodiment of the present invention;

FIG. 7 is a depiction of steps of a method of making a device according to one embodiment of the present disclosure.

### DETAILED DESCRIPTION

The drawings are purely schematic illustrations of various aspects of the invention and are not necessarily to scale, unless expressly stated.

The terms “substantially” or “about” used herein with reference to a quantity includes variations in the recited quantity that are equivalent to the quantity recited, such as an amount that is equivalent to the quantity recited for an intended purpose or function. “Substantially” or derivatives thereof will be understood to mean significantly or in large part.

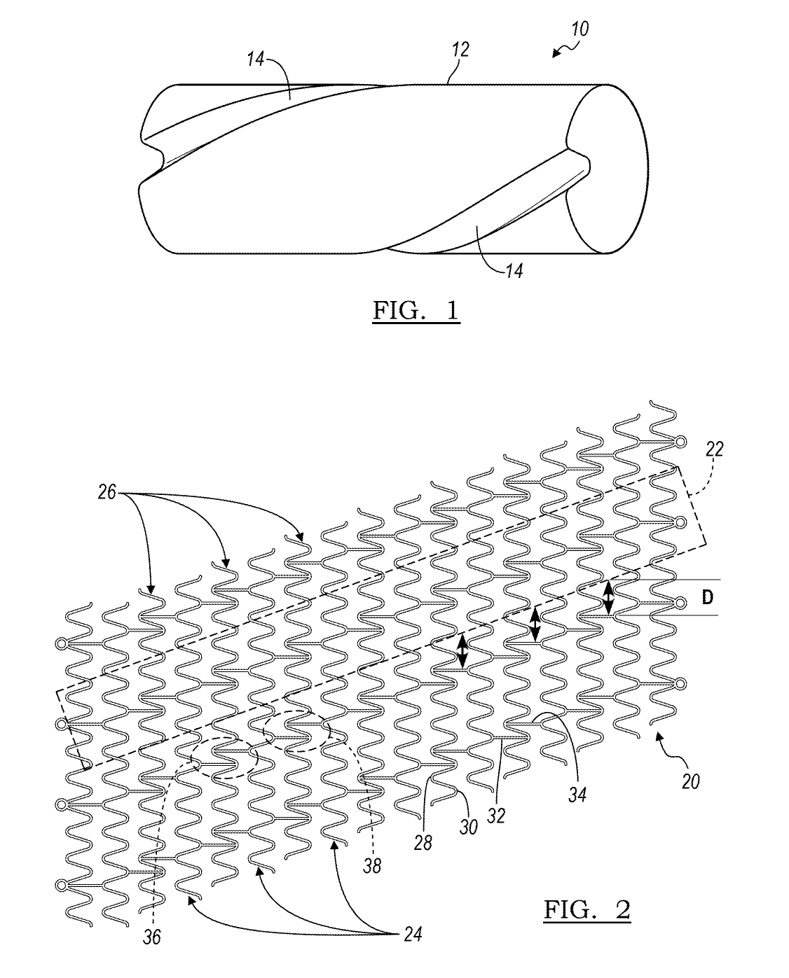

FIG. 1 depicts a cylindrical mandrel 10 which may be useful in constructing a device in accordance with the principles of the present disclosure. The mandrel 10 has an outer surface 12 which is largely cylindrical but which contains a groove 14 that winds about the length of the cylinder. Such a mandrel 10 can be used to describe, and to construct, a tubular implant, such as a stent, with a similarly grooved outer surface. However, in the case of a tubular device rather than a solid cylindrical mandrel, the grooved channel (or groove) 14 of the outer surface would impinge into the tubular lumen central to the device and give rise to an inner helical ridge.

The helical groove 14 and an interior ridge formed as a consequence of its presence could run for a portion of the length of a device, or the entire length of the device. The helical groove and ridge could have a helix angle ranging from about 10 degrees to about 45 degrees, or about 15 degrees to about 35 degrees, or about 20 degrees to about 30 degrees. Depending on the condition and/or vessel to be treated, the specific helix angle can be determined by a computation flow dynamics (CFD) analysis. A helix angle that is too small will fail to impart a spiral flow to the fluid passing through the device; one that is too large may cause greater turbulence to the flow. The terms “helix,” “helical,” and “helix angle” as used herein have their usual mathematical meanings. The ridge and groove may be a helical ridge and groove, or a spiral ridge and groove.

In some embodiments, the device will have a single helical groove. In other embodiments, it may be favorable to have a device that has a plurality of grooves formed therein.

FIG. 2 illustrates a flattened architecture for a device 20 constructed in accordance with the principles of the present disclosure. It will be appreciated that in designing a tubular device to be cut from a precursor cannula, a flattened plan may provide guidance. The pattern of device 20 provides for a position for groove 22, and is made of alternating flex rings 24 and hoop rings 26 connected to one another by a plurality of connectors 32 and 34.

In one embodiment, the rings (both hoop rings 26 and flex rings 24) may have an undulating or zigzag pattern, including a plurality of peaks 28 and valleys 30. The apices of certain of the peaks 28 and the valleys 30 are connected by connectors 32, 34 to an adjacent ring.

Each peak 28 and each valley 30 has an inner portion, between the two struts that converge to form the peak 28 or valley 30, and an outer portion, which is the “tip” or apex of the peak 28 or valley 30. When an outer portion of a peak 28 or valley 30 joins with a connector segment, the resulting shape is substantially like the letter Y. When an inner portion of a peak 28 or valley 30 is joined to a connector segment, the result is a W-shaped junction.

The rings of the device may be arranged as a plurality of struts and bends that give rise to a zigzag shape, appearing like a series of Vs connected together. The alternating, zigzag fashion means that the bends of the device are considered peaks and valleys. Whether a bend is considered a peak or a valley may be dependent on the perspective of the viewer, specifically whether the viewer is considering the device from the first end or from the second end. When an end is chosen, the bends of an ultimate ring of the device that define the extreme endpoints of the device are considered peaks, and the bends positioned between the peaks are considered valleys. This designation propagates to the other end of the device. Because of the symmetric nature of some embodiments of the device of the present disclosure, the bends defined as peaks when viewed from the first end will instead be considered valleys when viewed starting from the second end, and vice versa.

Additionally, each peak and each valley is a bend that has an inner portion and an outer portion. The inner portion of the peak or valley lies circumferentially between the two struts that adjoin the bend, and the outer portion lies longitudinally opposite of the inner edge. The outer edges represent the furthest extents, longitudinally, of the rings.

When adjacent rings have an in-phase relationship of struts and bends, an inner edge of one peak or valley, when attached to the adjacent ring by a tie bar or connector, will be attached to an outer edge of the corresponding structure on the adjacent ring.

Flex rings 24 are defined in that their connectors are on the outer portion, or apex, giving the Y shape. Hoop rings 26 instead are connected to adjacent flex rings 24 by connectors that contact their inner portion. This connection scheme holds true for connectors positioned at peaks 28 and at valleys 30 alike. Such an architecture also makes the flex rings 24 more flexible, and the hoop rings 26 more rigid.

An implant, such as a stent, which has the particular ring-and-connector configuration as is disclosed herein, has an alternating pattern of hoop rings and flex rings, such that besides the terminal rings, each hoop ring is disposed between two flex rings, and each flex ring is positioned between two hoop rings. Such a configuration, in a standard, non-grooved device, has a predictable geometry. Introducing the groove to a device agnostic of the overall strut and connector architecture can lead to a highly complex geometry in which every ring (or even portion of a ring) performs differently under loading and therefore is not predictable.

Prior to constructing the device 20, the pattern is defined to compensate for the presence of the groove. As shown in FIG. 2, a number of peak-to-peak connectors 34 connect hoop rings 26 to flex rings 24, and likewise a number of valley-to-valley connectors 32 connect adjacent rings to one another. The connector 32, 34 nearest the groove 22, as seen in FIG. 2, is a consistent distance D away from the groove 22, relative to a connector of another ring along the length of the device. The distance D will depend on a number of factors, including the circumference of the device. As a guideline, embodiments of the implant will include an equal or greater number of connectors between the rings as in a standard, non-grooved device, such as between three connectors and twelve connectors connecting a single flex ring to an adjacent hoop ring, and any number in between. The groove may contain connectors, or connectors may be absent from the groove. After the initial connectors closest to the groove are placed in the pattern for the device, the remaining connectors are then spaced circumferentially about the flex or hoop ring such that any one flex ring or hoop ring along the entire length of the device 20 will have a similar, or identical, pattern of connectors around the circumference, relative to the helical groove 22. The groove 22 has a helical angle as it winds around the device, and the connectors closest to the groove 22 are arranged along the length of the device to have the same helix angle, such that the pattern of connectors tracks the path of the groove.

As further seen in circles 36 and 38 of FIG. 2, connectors from one hoop ring 26 to the adjacent flex rings 24 on either side may occur in pairs, in one embodiment. In this case, the peak 28 of the hoop ring 26 is joined to the adjacent hoop ring 26 by a peak-to-peak connector 34. An adjacent valley 30 of the same hoop ring 26 is connected to the other adjacent flex ring 24 by a valley-to-valley connector 32. These paired connectors 36 contribute to the relative rigidity and radial stiffness profile of the hoop ring.

It will be noted that terminology referring to the positional relationship of various portions of the device, such as stating that a portion is “connected to” or “joined to” another element of the device, does not necessarily imply that these portions were separately formed and then attached together. Although this may be one way of constructing the device, in most embodiments the device will be cut (such as by a laser) from a precursor cannula.

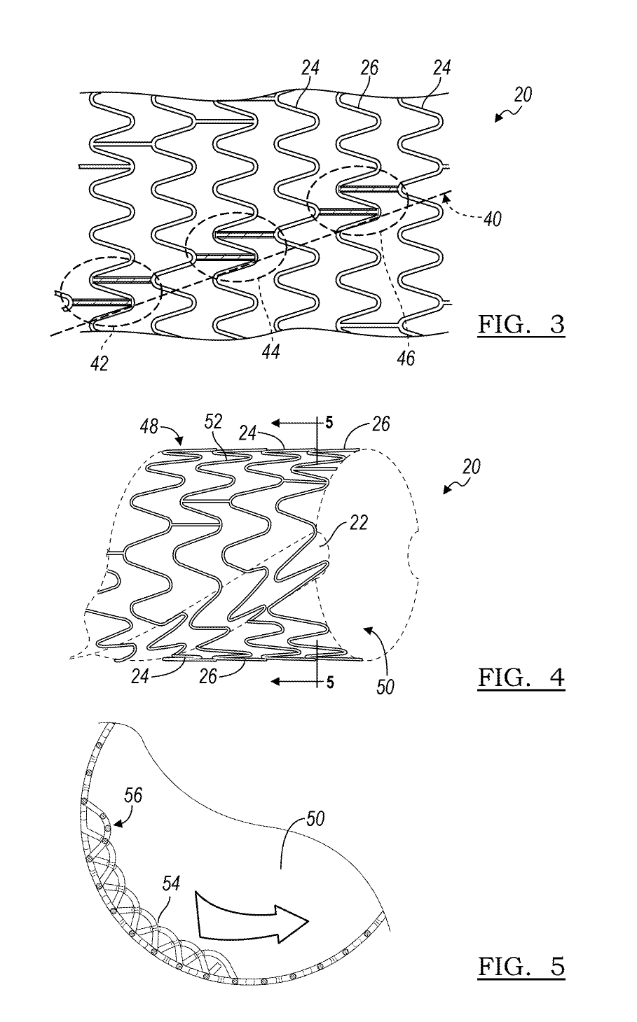

FIG. 3 illustrates a portion of the device 20 of FIG. 2 in its cylindrical form. Pairs 42, 44, 46 of connectors 32, 34 lie along a path 40 that tracks the helical profile of the groove 22. FIG. 4 provides a perspective view of device 20 rather than the side view of FIG. 3. In this view, outer surface 48 can be seen to form a substantially cylindrical profile 52 at positions away from the groove 22.

FIG. 5 is a cross sectional view of the device of FIG. 4 taken along line 5. In this view, the helical ridge 54 can clearly be seen in lumen 50 of the device. Because the ridge is formed of struts of zigzag rings pressed radially inwardly into the lumen, the ridge 54 can be seen as a series of arcs 56 when viewed from the end of the device. When blood (or another fluid) flows over the ridge, its motion takes on a spiral aspect.

As shown, the implant 20 is a substantially tubular element having an open first end and extending to an open second end such that fluid can flow in through one of said ends and out the other. The device 20 defines a longitudinal axis therethrough around which the rings are circumferentially arranged. The helical element presents as a groove on the outer surface of the device 20 and a ridge on the inner surface of the device.

Other embodiments of devices are in accordance with the principles of the present disclosure. For instance, the helical ridge/groove may originate at, or substantially at, the first end, and extend helically through the device to the second end. Alternatively, the helical element may begin or may terminate further along the length of the device, that is, away from one or both of the ends. In a particular embodiment, the ends of the device do not have a groove formed therein, such that fluid flow is not disrupted as fluid immediately enters or exits the lumen of the device. Rather, the fluid is treated within the middle 90%, or 80%, or 70% of the device with the introduction of a spiral flow pattern.

The helical groove may be formed at any depth suitable for the application. For example, the helical groove may have a depth of about 5% to 100% of the radius of the device, or from about 10% to about 50% of the radius. The groove may also be a relatively narrow groove, having a width representing about 2% of the circumference of the device, or may be larger, representing about 25% of the circumference of the device, or any value in between about 2% to about 25%. In certain embodiments, the width of the groove may represent about 7.5% of the circumference, or about 10% of the circumference, or about 12.5% of the circumference, or about 16.7% of the circumference. The groove is constructed with a width such that more than one strut is present, in part or completely, within the groove.

The device according to the principles of the present invention is a self-expanding device. Such a device may be made from a biocompatible material, or a material which is able to be made biocompatible. Examples of suitable materials include, without limitation, nickel-titanium alloys, cobalt-chromium alloys, nickel-chromium alloys, nickel-cobalt-chromium alloys, nickel-cobalt-chromium-molybdenum alloys, nickel-titanium-chromium alloys, and other shape memory and/or superelastic materials, including alloys which include at least one of molybdenum, tantalum, titanium, palladium, and platinum. Polymers and composite materials may also provide the properties necessary for making such a device.

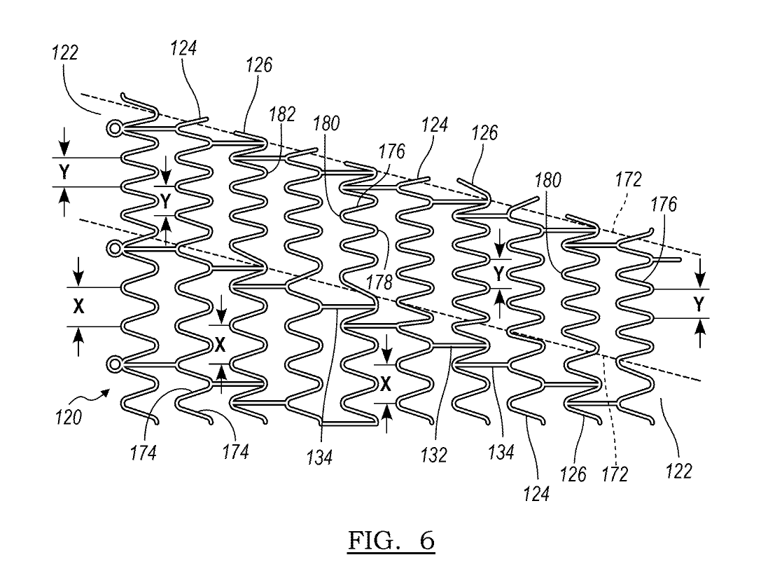

FIG. 6 illustrates another embodiment of a device 120 constructed in accordance with the principles of the present disclosure. The device 120 incorporates a variation on the flex ring 124/hoop ring 126 structure as discussed previously, but adds an additional dimension; namely, the device 120 has an increase in the density of the struts 176 inside the groove 122 as compared to the struts 174 located outside of the groove 122. It can be seen in FIG. 6 that a distance X between adjacent peaks outside of the groove boundaries 172 is greater than the distance between adjacent peaks 180 in the groove 122. An increase of struts 174 within the groove 122 provides more material to form the helical ridge in the lumen of the device which will have a greater impact on blood flow than struts having the usual spacing outside of the groove. The groove boundaries may be set between two sets of connectors to define the groove 122, and may lie on a series of connectors on either side of the groove. However, this does not preclude the presence of connectors within the groove itself.

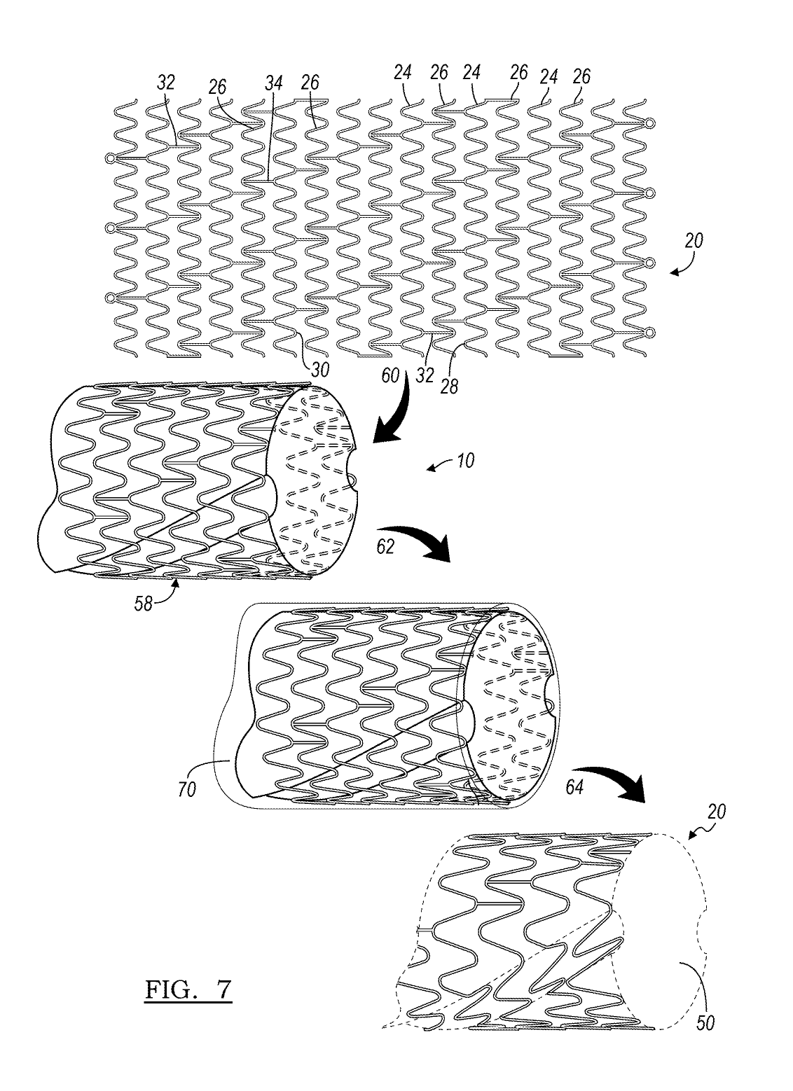

FIG. 7 illustrates a method of making a device in accordance with the principles of the present disclosure. First, a device 20 is cut from a cylindrical cannula by any suitable method. Laser cutting is particularly useful for this step. The device is cut in a pattern to form a plurality of ring structures each including a plurality of struts in a zigzag pattern. The ring structures are interconnected by a plurality of connector segments as in FIGS. 2-5, where the locations of the connector segments are chosen to align in a substantially helical or spiral pattern.

In a step 60, the cylindrical device 20 is placed over a grooved mandrel 58. The mandrel 58 has a groove which has the same helical angle as the arrangement of connectors in the device, such that when the device is placed over the mandrel 58, the distance between the connector of any given ring is spaced a substantially similar distance from the groove as one of any other corresponding ring of that type.

In step 62, a cylindrical sleeve is positioned over the device. The sleeve 70 forces the struts and connectors that overlay the groove of the mandrel 58 into the groove. In step 64, when portions of the device are fit into the groove, the device is heat set by modulating the temperature such that a remembered state is created, with the helical element being present in the remembered state.

When the device is complete, it may be loaded into a delivery assembly and implanted into a body vessel in need of support in which it is thought that regulation of blood flow may be required. The spiral flow of blood reduces turbulence and minimizes the chances that particulate will collect on or near the device. The device may be deployed similarly to other self-expanding intraluminal devices, and may be delivered over a wire guide. The device may include radiopaque markers such that the delivery can be monitored through visualization techniques, such as fluoroscopy.

Although the device as described herein has largely been characterized as a bare metal implant such as a stent, a grooved tubular device of this construction could form the basis for a number of different types of devices, such as a stent graft. To create a stent graft, the device might be provided with an inner liner of a biocompatible material, an outer liner, or both. The biocompatible material may be a woven polymer, or a polymer sheet, and may be attached to the struts and/or connectors of the device by any means known to be acceptable in the art.

A number of benefits may be realized from the incorporation of a helical ridge or element in the central lumen of a tubular device for modulating blood flow. The spiral flow that results may reduce the overall turbulence within the localized region being treated, and may reduce wall pressures and stresses. There may also be realized a reduction in particle adhesion to the vessel wall, and blood flow may be improved through a stenosis or blockage.

As a person skilled in the art will readily appreciate, the above description is meant as an illustration of implementation of the principles this application. This description is not intended to limit the scope of this application in that the system is susceptible to modification, variation and change, without departing from the spirit of this application, as defined in the following claims.

What is claimed is:

1. An intraluminal support device for implantation into a lumen of a body vessel, the intraluminal support device comprising:
a tubular body having a first end extending to a second end to define a length, the tubular body defining a longitudinal axis and defining a lumen, the tubular body having an inner surface and an outer surface, the first end and the second end being open and in fluid communication with the lumen,
the tubular body comprising a plurality of rings disposed axially along the longitudinal axis, each ring being connected to an adjacent ring by at least one connector segment, the tubular body being radially expandable from a compressed state to an expanded state, a plurality of the connector segments being arranged in a helical pattern having a helical angle,
the intraluminal support device including a helical groove formed in the tubular body and defining a helical ridge extending into the lumen, the helical groove having a helical angle equal to the helical angle of the helical pattern, the connector segments of the helical pattern being spaced substantially constantly from the groove along the length of the tubular body.

2. The intraluminal support device of claim 1, wherein the helical angle is about 90 degrees to about 360 degrees about a circumference of the tubular body.

3. The intraluminal support device of claim 1, wherein each of the plurality of rings comprise a plurality of struts and bends arranged in a repeating pattern to define a plurality of peaks and a plurality of valleys.

4. The intraluminal support device of claim 3, comprising a plurality of hoop rings wherein connector segments connect at peaks, and a plurality of flex rings wherein connector segments connect at valleys.

5. The intraluminal support device of claim 4, wherein the hoop rings and the flex rings are disposed in alternating fashion axially.

6. The intraluminal support device of claim 4, wherein a connector segment outside of and nearest to the helical groove of a first hoop ring is located substantially equidistant circumferentially from the helical groove as a corresponding connector nearest to the helical groove of a second hoop ring.

7. The intraluminal support device of claim 1, wherein a peak-to-peak connector segment nearest the groove is paired with an adjacent valley-to-valley connector segment.

8. The intraluminal support device of claim 1, wherein the intraluminal support device is self-expanding.

9. The intraluminal support device of claim 7, wherein the intraluminal support device comprises a shape-memory material.

10. The intraluminal support device of claim 8, wherein the shape memory material comprises a nickel-titanium alloy.

11. The intraluminal support device of claim 1, wherein the density of connector segments is higher nearer the groove than away from the groove.

12. The intraluminal support device of claim 7, wherein the tubular body is cut from a unitary cylinder.

13. The intraluminal support device of claim 1, wherein a distance between adjacent peaks of a ring within the helical groove is less than a distance between adjacent peaks of the ring outside of the helical groove.

14. An intraluminal support device for implantation into a lumen of a body vessel, the intraluminal support device comprising:
a tubular body having a first end extending to a second end and defining a length, the tubular body defining a longitudinal axis and defining a lumen, the tubular body having an inner surface and an outer surface, the first end and the second end being open and in fluid communication with the lumen,
the tubular body comprising a plurality of hoop rings and flex rings disposed axially and alternately along the longitudinal axis, each flex ring being connected to an adjacent hoop ring by at least one connector segment, the tubular body being radially expandable from a compressed state to an expanded state, a plurality of the connector segments being arranged in a helical pattern having a helical angle,
the intraluminal support device including a helical groove formed in the tubular body and defining a helical ridge extending into the lumen, the helical groove having a helical angle equal to the helical angle of the helical pattern, the connector segments of the helical pattern being spaced substantially constantly from the groove along the length of the tubular body.

15. The intraluminal support device of claim 14, wherein the helical groove has a helical angle of about 90 degrees to about 360 degrees about a circumference of the tubular body.

16. The intraluminal support device of claim 14, wherein each of the connector segments are substantially parallel to the longitudinal axis in the compressed state.

17. The intraluminal support device of claim 14, wherein the intraluminal support device is self-expanding.

18. The intraluminal support device of claim 14, wherein the device comprises a shape memory material.

19. The intraluminal support device of claim 14, wherein the density of connector segments is higher nearer the groove than away from the groove.

20. The intraluminal support device of claim 14, wherein a distance between adjacent peaks of a ring within the helical groove is less than a distance between adjacent peaks of the ring outside of the helical groove.

21. A method of making an intraluminal support device for implantation into a body vessel, the method comprising:
placing the intraluminal support device over a cylindrical mandrel having a helical groove formed thereon, the intraluminal support device comprising:
a tubular body having a first end extending to a second end and defining a length, the tubular body defining a longitudinal axis and a lumen, the tubular body having an inner surface and an outer surface, the first end and the second end being open and in fluid communication with the lumen, the tubular body being radially expandable from a compressed state to an expanded state,
the tubular body comprising a plurality of rings disposed axially along the longitudinal axis, each of the rings being connected to one another by at least one connector segment to form a plurality of rings, a plurality of the connector segments being arranged in a helical pattern, the connector segments of the helical pattern being spaced substantially constantly from the groove along the length of the tubular body;
securing the intraluminal support device to the cylindrical mandrel such that the helical groove of the mandrel is bounded by the connector segments of the helical pattern;
fitting a portion of each ring into the helical groove; and
heat-setting the intraluminal support device such that the outer surface includes a helical groove formed therein and extending into the lumen.
